(19) United States
(12) Patent Application Publication
(10) Pub. No.: US 2004/0133360 A1
(43) Pub. Date: Jul. 8, 2004

(54) Method and computer software product for defining multiple probe selection regions

(75) Inventors: Ray Wheeler, Berkeley, CA (US); Simon Cawley, Oakland, CA (US); David Kulp, Albany, CA (US); Alan Williams, Albany, CA (US); Brant Wong, Walnut Creek, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/738,546

(22) Filed: Dec. 16, 2003

Related U.S. Application Data
(63) Continuation of application No. 10/027,682, filed on Dec. 21, 2001, now abandoned.

(51) Int. Cl. C12Q 1/68; G06F 19/00; G01N 33/48; G01N 33/50

(52) U.S. Cl. 702/20; 435/6

(57) ABSTRACT

In one embodiment of the invention, methods are provided for selecting probes upstream from alternative polyadenylation sites of a target sequence cluster.

### RELATED APPLICATIONS

This application is related to U.S. patent application Ser. No. 09/721,042, filed on Nov. 21, 2000, entitled “Methods and Computer Software Products for Predicting Nucleic Acid Hybridization Affinity”; U.S. patent application Ser. No. 09/718,295, filed on Nov. 21, 2000, entitled “Methods and Computer Software Products for Selecting Nucleic Acid Probes” and U.S. patent application Ser. No. 09/745,965, filed on Dec. 21, 2000, entitled “Methods For Selecting Nucleic Acid Probes”; U.S. patent application Ser. No. 10/006,174, filed on Dec. 4, 2001, and U.S. patent application Ser. No. ______, attorney Docket No. 3439, filed on Dec. 21, 2001, and U.S. patent application Ser. No. ______, attorney docket number 3441, filed on Dec. 21, 2001. All the cited applications are incorporated herein by reference in their entireties for all purposes.

### BACKGROUND OF THE INVENTION

This invention is related to bioinformatics and biological data analysis. Specifically, this invention provides methods, computer software products and systems for determining the orientation of biological sequence clusters. In some embodiments, the methods, computer software products and systems are used for designing nucleic acid probe arrays.

Typically, the nucleic acid probe array design involves the definition of regions in targets the probes may detect. For example, oligonucleotide probes on high density gene expression monitoring probe arrays are typically designed to hybridize with specific regions of transcripts or nucleic acids derived from the specific regions of transcripts.

### SUMMARY OF THE INVENTION

In one aspect of the invention, methods for designing a nucleic acid probe array to target a transcript cluster. The methods include selecting a first set of probes comprising at least one probe targeting a first region immediately upsteam of a first polyadenylation site; and selecting a second set of probes comprising at least one probe targeting a second region immediately upstreatm of a second polyadenylation site, wherein the first and second regions are different. The first and second polyadenylation sites are alternative polyadenylation sites. The first polyadenylation site can be a putative or predicted polyadenylation site. Typically, the first region is within 600, 800 bases upstream of the first polyadenylation site and the second region is within 600, 800 bases upstream of the second polyadenylation site.

The transcript cluster can include RNA and EST sequences. If the first polyadenylation site is in a full length mRNA, the first set of probes are selected to target the full length mRNA as an exemplar sequence of the cluster. The first polyadenylation site is shared by a stack of sequences in the cluster and the probe are selected to target the consensus sequence of the cluster, wherein the stack of sequences comprises at least 2, 6, 8, 10 sequences.

In another aspect of the invention, nucleic acid probe arrays with probes selected according to the methods of the invention are provided. Exemplary arrays include high density oligonucleotide arrays or spotted arrays. The probes are typically immobilized at a density of 400, 1000, 10000 different probes per cm².

In another aspect of the invention, systems and computer software are provided for performing the methods of the invention. The systems include a processor; and a memory coupled with the processor, the memory storing a plurality of machine instructions that cause the processor to perform logical steps of the methods of the invention. The computer software products of the invention include a computer readable medium having computer-executable instructions for performing the method of the invention.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 is a schematic showing an exemplary computer system suitable for executing some embodiments of the software of the invention.

FIG. 2 is a schematic showing the architecture of the exemplary computer system of FIG. 1.

FIG. 3 shows an exemplary computer network system suitable for executing some embodiments of the software of the invention.

FIG. 4 shows a process for designing a nucleic acid probe array.

FIG. 5 shows a sequence with multiple alternative feature sites.

FIG. 6 shows a multiple sequence selection region.

FIG. 7 shows an example of alternative polyadenylation.

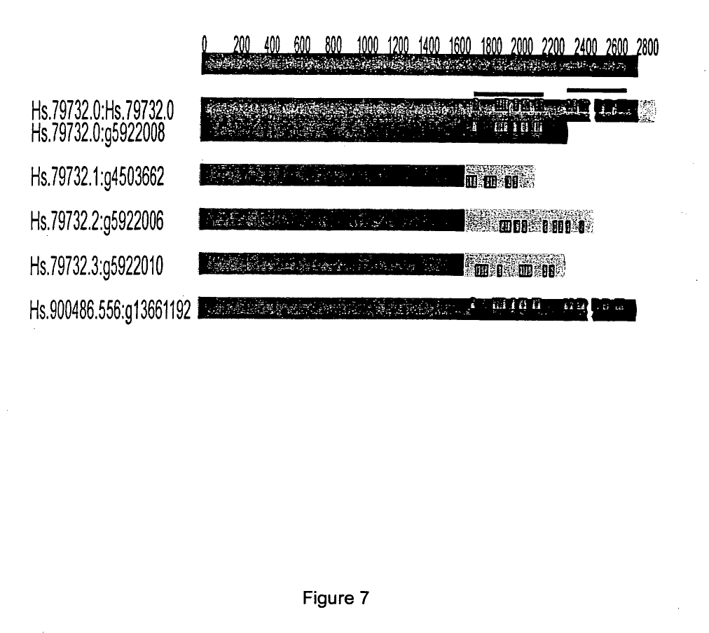

### DETAILED DESCRIPTION

Reference will now be made in detail to the exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Throughout this disclosure, various aspects of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention will employ, unless otherwise indicated, conventional techniques of bioinformatics, computer sciences, immunology, biochemistry, chemistry, molecular biology, microbiology, cell biology, genomics and recombinant DNA, which are within the skill of the art. See, e.g., Setubal and Meidanis, et al., 1997, Introduction to Computational Molecular Biology, PWS Publishing Company, Boston; Human Genome Mapping Project Resource Centre (Cambridge), 1998, Guide to Human Genome Computing, 2nd Edition, Martin J. Biship (Editor), Academic Press, San Diego; Salzberg, Searles, Kasif, (Editors), 1998, Computational Methods in Molecular Biology, Elsevier, Amsterdam; Matthews, PLANT VIROLOGY, 3^{rd} edition (1991); Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987))

System for Sequence Annotation and for Nucleic Acid Probe Array Design

In aspects of the invention, methods, computer software and systems for determining the orientation of EST (cDNA?) sequence clusters and for probe array design are provided. One of skill in the art would appreciate that many computer systems are suitable for carrying out the methods of the invention. Computer software according to the embodiments of the invention can be executed in a wide variety of computer systems.

For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems: A Practical Guide for Systems Professionals by Paul E. Renaud, 2nd edition (June 1996), John Wiley & Sons; ISBN: 0471133337, both are incorporated herein by reference in their entireties for all purposes.

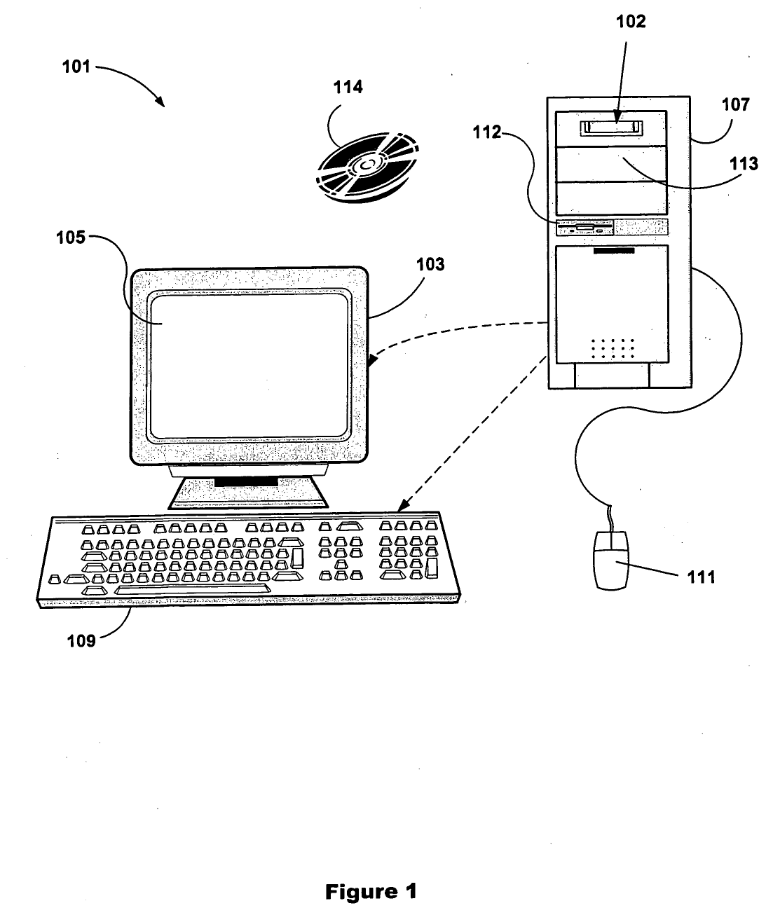

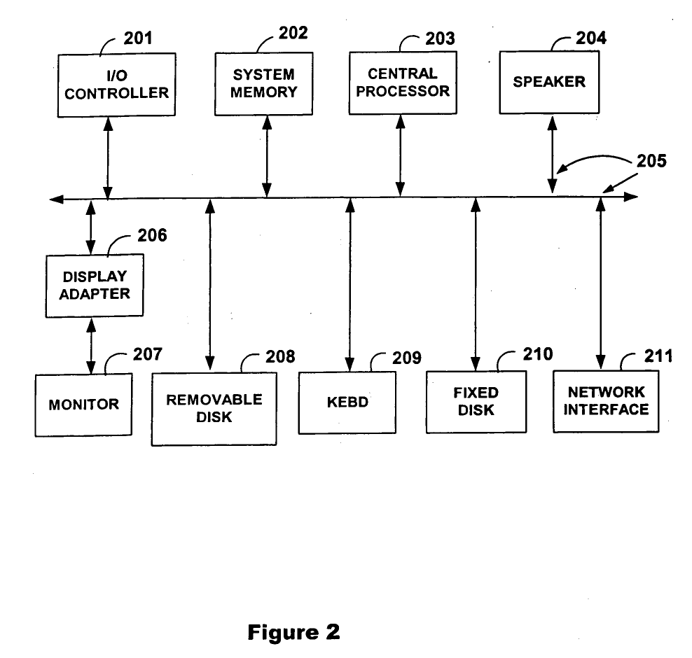

FIG. 1 illustrates an example of a computer system that may be used to execute the software of an embodiment of the invention. FIG. 1 shows a computer system 101 that includes a display 103, screen 105, cabinet 107, keyboard 109, and mouse 111. Mouse 111 may have one or more buttons for interacting with a graphic user interface. Cabinet 107 houses a floppy drive 112, CD-ROM or DVD-ROM drive 102, system memory and a hard drive (113) (see also FIG. 2) which may be utilized to store and retrieve software programs incorporating computer code that implements the invention, data for use with the invention and the like. Although a CD 114 is shown as an exemplary computer readable medium, other computer readable storage media including floppy disk, tape, flash memory, system memory, and hard drive may be utilized. Additionally, a data signal embodied in a carrier wave (e.g., in a network including the Internet) may be the computer readable storage medium.

FIG. 2 shows a system block diagram of computer system 101 used to execute the software of an embodiment of the invention. As in FIG. 1, computer system 101 includes monitor 201, and keyboard 209. Computer system 101 further includes subsystems such as a central processor 203 (such as a Pentium™ III processor from Intel), system memory 202, fixed storage 210 (e.g., hard drive), removable storage 208 (e.g., floppy or CD-ROM), display adapter 206, speakers 204, and network interface 211. Other computer systems suitable for use with the invention may include additional or fewer subsystems. For example, another computer system may include more than one processor 203 or a cache memory. Computer systems suitable for use with the invention may also be embedded in a measurement instrument.

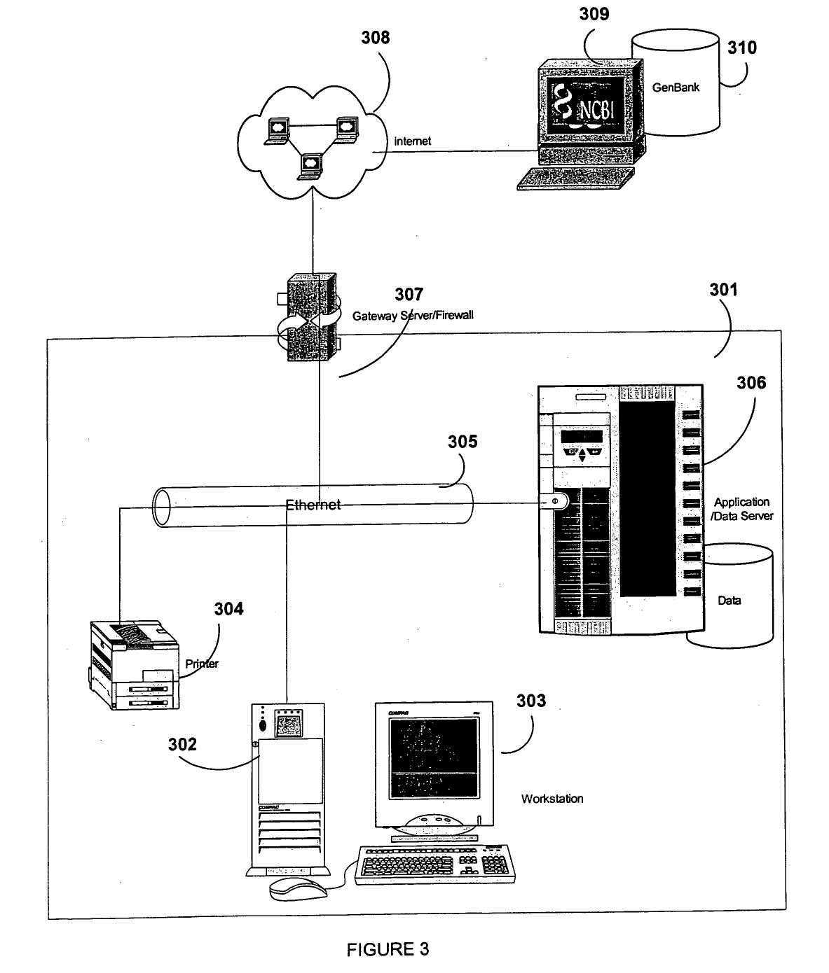

FIG. 3 shows an exemplary computer network that is suitable for executing the computer software of the invention. A computer workstation 302 is connected with the application/data server(s) through a local area network (LAN) 301, such as an Ethernet 305. A printer 304 may be connected directly to the workstation or to the Ethernet 305. The LAN may be connected to a wide area network (WAN), such as the Internet 308, via a gateway server 307 which may also serve as a firewall between the WAN 308 and the LAN 305. In some embodiments, the workstation may communicate with outside data sources, such as the National Biotechnology Information Center, through the Internet. Various protocols, such as FTP and HTTP, may be used for data communication between the workstation and the outside data sources. Outside genetic data sources, such as the GenBank 310, are well known to those skilled in the art. An overview of GenBank and the National Center for Biotechnology information (NCBI) can be found in the web site of NCBI (“www.ncbi.nlm.nih.gov”).

Computer software products of the invention typically include computer readable medium having computer-executable instructions for performing the logic steps of the methods of the invention. Suitable computer readable medium include floppy disk, CD-ROM/DVD/DVD-ROM, hard-disk drive, flash memory, ROM/RAM, magnetic tapes and etc. The computer executable instructions may be written in any suitable computer language or combination of several languages. Suitable computer languages include C/C++ (such as Visual C/C++), C#, Java, Basic (such as Visual Basic), SQL, Fortran, SAS and Perl.

Nucleic Acid Probe Arrays

The methods, computer software and systems of the invention are particularly useful for designing high density nucleic acid probe arrays.

High density nucleic acid probe arrays, also referred to as “DNA Microarrays,” have become a method of choice for monitoring the expression of a large number of genes and for detecting sequence variations, mutations and polymorphism. As used herein, “nucleic acids” may include any polymer or oligomer of nucleotides or nucleotides (polynucleotides or oligonucleotidies), which include pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982) and L. Stryer, BIOCHEMISTRY, 4^{th} Ed. (March 1995), both incorporated by reference. “Nucleic acids” may include any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

“A target molecule” refers to a biological molecule of interest. The biological molecule of interest can be a ligand, receptor, peptide, nucleic acid (oligonucleotide or polynucleotide of RNA or DNA), or any other of the biological molecules listed in U.S. Pat. No. 5,445,934 at col. 5, line 66 to col. 7, line 51, which is incorporated herein by reference for all purposes. For example, if transcripts of genes are the interest of an experiment, the target molecules would be the transcripts. Other examples include protein fragments, small molecules, etc. “Target nucleic acid” refers to a nucleic acid (often derived from a biological sample) of interest. Frequently, a target molecule is detected using one or more probes. As used herein, a “probe” is a molecule for detecting a target molecule. It can be any of the molecules in the same classes as the target referred to above. A probe may refer to a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners. When referring to targets or probes as nucleic acids, it should be understood that these are illustrative embodiments that are not to limit the invention in any way.

In some embodiments, probes may be immobilized on substrates to create an array. An “array” may comprise a solid support with peptide or nucleic acid or other molecular probes attached to the support. Arrays typically comprise a plurality of different nucleic acids or peptide probes that are coupled to a surface of a substrate in different, known locations. These arrays, also described as “microarrays” or colloquially “chips” have been generally described in the art, for example, in Fodor et al., Science, 251:767-777 (1991), which is incorporated by reference for all purposes. Methods of forming high density arrays of oligonucleotides, peptides and other polymer sequences with a minimal number of synthetic steps are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668, U.S. Pat. Nos. 5,677,195, 5,800,992 and 6,156,501, which disclose methods of forming vast arrays of peptides, oligonucleotides and other molecules using, for example, light-directed synthesis techniques. See also, Fodor, et al., Science, 251, 767-77 (1991). These procedures for synthesis of polymer arrays are now referred to as VLSIPS™ procedures.

Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743 and 6,140,044, all of which are incorporated by reference in their entireties for all purposes.

Microarray can be used in a variety of ways. An exemplary microarray contains nucleic acids and is used to analyze nucleic acid samples. Typically, a nucleic acid sample is prepared from appropriate source and labeled with a signal moiety, such as a fluorescent label. The sample is hybridized with the array under appropriate conditions. The arrays are washed or otherwise processed to remove non-hybridized sample nucleic acids. The hybridization is then evaluated by detecting the distribution of the label on the chip. The distribution of label may be detected by scanning the arrays to determine fluorescence intensity distribution. Typically, the hybridization of each probe is reflected by several pixel intensities. The raw intensity data may be stored in a gray scale pixel intensity file. The GATC™ Consortium has specified several file formats for storing array intensity data. The final software specification is available at www.gatcconsortium.org and is incorporated herein by reference in its entirety. The pixel intensity files are usually large. For example, a GATC™ compatible image file may be approximately 50 Mb if there are about 5000 pixels on each of the horizontal and vertical axes and if a two byte integer is used for every pixel intensity. The pixels may be grouped into cells (see, GATC™ software specification). The probes in a cell are designed to have the same sequence (i.e., each cell is a probe area). A CEL file contains the statistics of a cell, e.g., the 75th percentile and standard deviation of intensities of pixels in a cell. The 50, 60, 70, 75 or 80th percentile of pixel intensity of a cell is often used as the intensity of the cell.

Nucleic acid probe arrays have found wide applications in gene expression monitoring, genotyping and mutation detection. For example, massive parallel gene expression monitoring methods using nucleic acid array technology have been developed to monitor the expression of a large number of genes (e.g., U.S. Pat. Nos. 5,871,928, 5,800,992 and 6,040,138; de Saizieu et al., 1998, Bacteria Transcript Imaging by Hybridization of total RNA to Oligonucleotide Arrays, NATURE BIOTECHNOLOGY, 16:45-48; Wodicka et al., 1997, Genome-wide Expression Monitoring in Saccharomyces cerevisiae, NATURE BIOTECHNOLOGY 15:1359-1367; Lockhart et al., 1996, Expression Monitoring by Hybridization to High Density Oligonucleotide Arrays. NATURE BIOTECHNOLOGY 14:1675-1680; Lander, 1999, Array of Hope, NATURE-GENETICS, 21(suppl.), at 3, all incorporated herein by reference for all purposes). Hybridization-based methodologies for high throughput mutational analysis using high-density oligonucleotide arrays (DNA chips) have been developed, see Hacia et al., 1996, Detection of heterozygous mutations in BRCA1 using high density oligonucleotide arrays and two-color fluorescence analysis. Nat. Genet. 14:441-447, Hacia et al., New approaches to BRCA1 mutation detection, Breast Disease 10:45-59 and Ramsey 1998, DNA chips: State-of-Art, Nat Biotechnol. 16:40-44, all incorporated herein by reference for all purposes). Oligonucleotide arrays have been used to screen for sequence variations in, for example, the CFTR gene (U.S. Pat. No. 6,027,880, Cronin et al., 1996, Cystic fibrosis mutation detection by hybridization to light-generated DNA probe arrays. Hum. Mut. 7:244-255, both incorporated by reference in their entireties), the human immunodeficiency virus (HIV-1) reverse transcriptase and protease genes (U.S. Pat. No. 5,862,242 and Kozal et al., 1996, Extensive polymorphisms observed in HIV-1 dade B protease gene using high density oligonucleotide arrays. Nature Med. 1:735-759, both incorporated herein by reference for all purposes), the mitochondrial genome (Chee et al., 1996, Accessing genetic information with high density DNA arrays. Science 274:610-614) and the BRCA1 gene (U.S. Pat. No. 6,013,449, incorporated herein by reference for all purposes).

Methods for signal detection and processing of intensity data are additionally disclosed in, for example, U.S. Pat. Nos. 5,445,934, 547,839, 5,578,832, 5,631,734, 5,800,992, 5,856,092, 5,936,324, 5,981,956, 6,025,601, 6,090,555, 6,141,096, 6,141,096, and 5,902,723. Methods for array based assays, computer software for data analysis and applications are additionally disclosed in, e.g., U.S. Pat. Nos. 5,527,670, 5,527,676, 5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252, 5,834,758, 5,837,832, 5,843,655, 5,856,086, 5,856,104, 5,856,174, 5,858,659, 5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174, 5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996 and 6,136,269, all of which are incorporated by reference in their entireties for all purposes.

Nucleic Acid Probe Array Design Process

In some embodiments, a nucleic acid probe array design process involves selecting the target sequences and selecting probes. For example, if the probe array is designed to detect the expression of genes at the transcript level. The target sequences are typically transcript sequences. Selection of the target sequence may involve the characterization of the target sequence based upon available information. For example, expressed sequence tags information needs to be assembled and annotated.

After target sequences are identified, probes for detecting the target sequences can be selected. The probe sequences and layout information are then translated to photolithographic masks, commands for controlling ink-jet directed synthesis, or soft lithographic synthesis process.

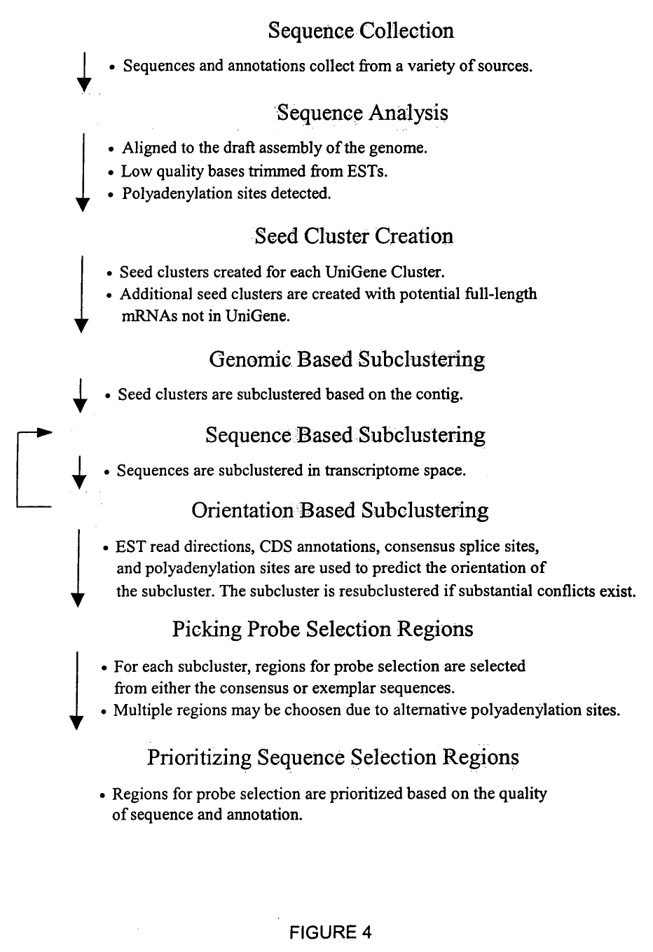

FIG. 4 shows one exemplary process for desiging a gene expression probe array.

SEQUENCE COLLECTION AND ANALYSIS. To provide the most complete starting information, cDNA sequence data can be obtained from primary sequence sources such as: GenBank, RefSeq dbEST and Washington University (WUSTL). Sequence meta information such as descriptions and definitions, clone identifiers, library identifiers, read directions, CDS annotations, low quality base annotations, gene names, and gene products can be extracted from the external data files in addition to the actual sequence.

In some instances, all input sequences are aligned to the assembly of the human genome. Typically, high quality regions of genome alignment are used to annotate and analyze the input sequences. The genomic alignments can also be used to confirm sequence orientation and consensus splice sites for many sequences.

In some instances, low quality EST sequence regions may be identified and removed according to the following exemplary rules:

1. Trim according to sequence quality annotation in dbEST report files.

2. Submissions from “The Institute for Genomic Research” and “Genethon” are considered already trimmed.

3. Sequences with Wash-U quality scores are considered already trimmed.

4. Trim according to genomic alignment with at least 90% identity across the entire EST or the alignment block of at least 180 bases.

5. Sequences from groups submitting less than 100 sequences are considered trimmed.

6. Trim from the right side to the mean high quality length for submissions in that year. Sequences before 1995 have been considered pre-trimmed.

These approaches reduced the presence of low quality bases, which may disrupt the clustering process and potentially contaminate the sequence content on the array.

POLYADENYLATION SITES. Messenger RNA is most efficiently converted into labeled target when it is adjacent to a poly-A site. Therefore, it is sometime important to dentify polyadenylation sites, since probes are generally selected within 600 bp upstream of the site, if the expression assays are 3′ biased. The use of untrimmed, primary sequence information helps significantly in this regard because poly-A or poly-T tracts are often removed prior to submission to public databases. Polyadenylation sites may be identified and a site score was calculated using a heuristic that accounts for the length of the poly-A (or poly-T, 5′ read), the amount of 5′ (or 3′) extraneous sequence, and the degree of interruption within the poly-A (or poly-T) (See, U.S. patent application Ser. No. ______, Attorney Docket Number 3439). For those sequences with a polyadenylation site, the presence of a polyadenylation signal may be determined using a probabilistic model.

VECTOR CONTAMINATION AND REPEATS. Sequences may be assessed for repeats, e.g., using RepeatMasker software and for vector contamination using, e.g., BLASTN and the UniVector database.

GENOME BASED SUBCLUSTERING. In a number of cases a UniGene cluster represents several genes within a gene family. Genome based subclustering is applied using the alignment information for each member sequence to the genomic sequence. Sequences that aligned to different contigs are assigned to separate subclusters. Those sequences that did not align to the genomic sequence are added to the largest subcluster.

SEQUENCE BASED SUBCLUSTERING. At this time, the human genome assembly remains incomplete and the quality is highly variable. It is therefore still necessary to refine seed clusters using a transcriptome based clustering approach. This was accomplished using the Cluster and Alignment Tool (CAT). To be conservative in selecting probes, 75 percent identity in all of the member sequences is required when a consensus is called. This eliminates problems with ambiguous and polymorphic bases.

ORIENTATION BASED SUBCLUSTERING. Subcluster orientation is determined using information from the following:

1. Sequence-label information, such as CDS annotations and read directions are used in the determination. In cases where introns are clearly delineated, consensus splice-site flanking sequences are used for orientation determination.

2. The intron flanking sequence GT-AG, AT-AC, or GC-AG indicates the sense orientation while CT-AC, GT-AT, or CT-GC implies an anti-sense orientation.

3. Polyadenylation signals and sites (5′ stretches of T's or 3′ stretches of A's) also provided orientation information.

A combination of the above information is used to make an orientation call of sense, anti-sense, or unknown for each member sequence used. Clusters with a problematic orientation were resubclustered by placing all the sequence members with evidence of a sense orientation into one subcluster and all the members with evidence of an anti-sense orientation into another subcluster. Sequences with an unknown orientation were placed into the larger of these.

PROBE SELECTION REGIONS. One of skill in the art would appreciate that particular sequence features, such as a polyadenylation site, may reside in several possible locations in a sequence or a sequence cluster. For example, a given subcluster, while typically representing one transcript variant, may represent several alternative polyadenylation sites. In one aspect of the invention, arrays that have probes selected from several regions are provided. The multiple sequene selection regions are used to ensure that at least one probe set is targeting the region close to a polyadenylation site. In some instances, sequence features, such as a polyadenylation site, may be tentative or putative, particlulary in cases of computationally predicted sequence features. In some embodiments, probes are selected to target the tentative or putative features.

In a transcript cluster, there are typically a number of RNA or EST sequences. As used herein, the term “transcript” refers to a RNA transcript. Term “transcript cluster” is a cluster of EST, RNA sequences or other sequences representing an actual or putative transcript. Projects related to EST clustering and assembly include UniGene from the National Center for Biotechnology Information; the TIGR Gene Index (http://www.tigr.org/tdb/hgi/hgi.html) from the Institute for Genomic Research; the Sequence Tag Alignment and Consensus Knowledgebase (STACK; http://ziggy.sanbi.ac.za/stack/stacksearch.htm); the Merck/Washington University Gene Index; and the GenExpress project. All of these projects perform some type of cluster analysis in which sequence similarity is used to form the clusters. For an overview of EST and RNA clustering, see, Win Hide and Alan Christoffels, EST Clustering Tutorial, ISMB, 1999 (available at www.sanbi.ac.za) and incorporated here by reference. It is worth noting that the gene indexing process typically incorporate information about EST and full length cDNA sequences.

One method that can be used to identify a polyadenylation site in a transcript cluster is to analyze the multiple alignment results from the CAT clustering tool. Candidates for a polyadenylation site are the aligned 3′ ends of full length transcripts with 3′ UTR and aligned “stacks” of 3′ EST's. Full length transcripts are typically any sequence from RefSeq, or a GenBank sequence annotated with complete CDS. These sequences are determined to have 3′ UTR if the 3′ end of the CDS annotation were greater than 5, 7, 10, 12, 20 bases away from the end of the sequence. An aligned “stack” of 3′ EST's was defined to be a group of EST's such that the positions of each EST's 3′ alignment end was no more than 5, 10, 15, 20, 25 aligned bases away from its nearest neighbor. In cases where 2 or more full length transcripts with 3′ UTR or aligned “stacks” of 3′ EST' are less than 300, 350, 400, 450, 500, 550, or 600 the more 5′ end was chosen so that probes for that region might be able to detect both polyadenylation sites.

In preferred embodiments, a group of nucleic acid probes are provided for gene expression monitoring. The probe group includes a first set of probes against a first region of a transcript cluster; and a second set of probes against a second region of the transcript cluster, wherein the first region is within 800 base pair upstream of a first actual or putative polyadenylation site and the second region is within 800 base pair upstream of a second actual or putative polyadenylation site.

In some embodiments, the first region is within 400 or 600 base pair upstream of the first actual or putative polyadenylation site and the second region is within 400 or 600 base pair upstream of a second actual or putative polyadenylation site.

The nucleic acid probes may be oligonucleotides or analogues of oligonucleotides from 8-80 bases long, preferably 28-30 bases long. The oligonucleotides may be immobilized on a solid surface such as a flate substrate to form a nucleic acid probe array. The oligonucleotides can also be immobilized in beads where it is preferred to immobilize one type of probe per bead. As used herein, the term “one probe” or “a probe” refers to one type of probes. Therefore, one probe can have many molecules of the same probe. In some instances, the molecules of a probe may have different lengths. For example, when a probe is synthesized on a location on a substrate as in an array, the molecules on the same location may have somewhat varied lengths. However, all the molecules of the same probe are designed to hybridize with their intended target.

Typically, each probe set has at least 1, 2, 3, 5, 10, or 20 probes. In some instances, the probes are paired to form mismatch/prefect match pair. The probe pair arrangements are particularly useful for gene expression monitoring.

In preferred embodiments, the many probe groups, at least 100, 500, 1000 groups of probes may be immobilized on a substrate at a density of greater than 400, 1000, 10000, 300000 different probes per cm². In some instances, some of the groups may not have multipe sets of probes targeting alternative regions. For instance, an oligonucleotide probe array may have a probe group against a transcript with a single known polyadenylation site. The same array may also have a probe group with 2, 3,4, or more sets of probes, each of the sets of probes targets a region upstream of an alternative polyadenylation site.

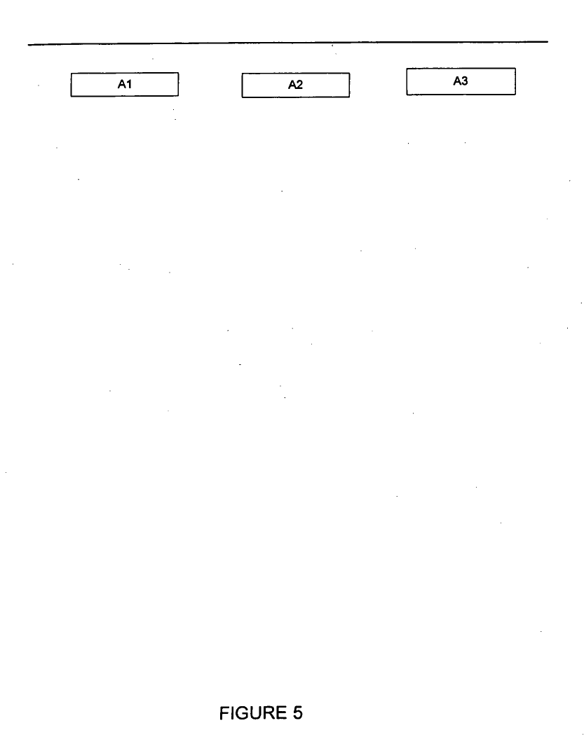

Based on the orientation call, the 3′ end of the cluster is identified. For clusters of unknown or ambiguous orientation, probes were picked against both ends of the sequence. Potential transcript ends are identified by the 3′ end of a potential full length member sequence, by a set of 5, 8, 10, 12 or more EST ends (5′ end of a 3′ EST or a polyadenylated EST), or by the end of the consensus sequence (FIG. 5). A 200-1000 bases region (400-800, 500-700, or 600) base region upstream of the end is chosen for probe selection.

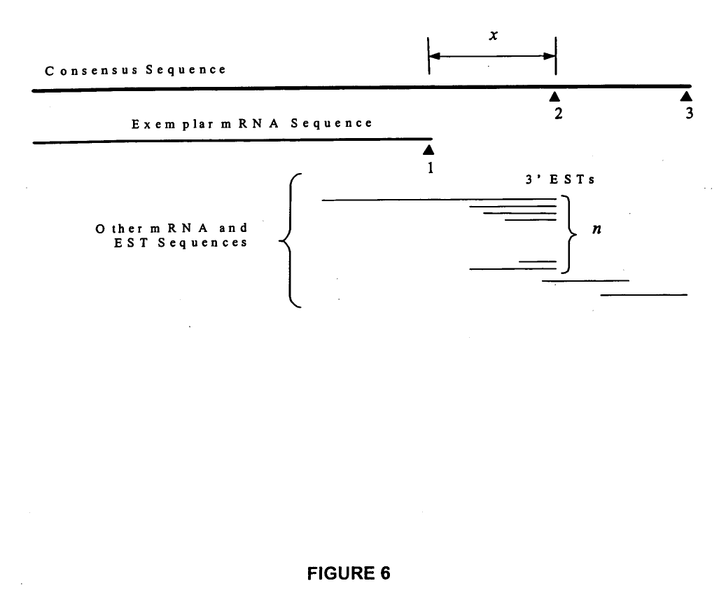

For putative transcript ends based on a potential full length mRNA, the corresponding mRNA sequence is used as an exemplar when picking probes. For all other transcript ends, the consensus sequence is used. A consequence of this strategy is that there can be multiple probe sets representing a particular sequence (FIG. 6).

FIG. 6 represents multiple pair-wise alignments to the seed sequence, Hs79732.0. The first portion of each label indicates the subcluster and the second portion indicates the sequence. Consensus sequences start with ‘Hs’ while exemplar sequences start with ‘g’. The two small bars at the top indicate the span of each probe set that detects the seed sequence. Dark regions are strongly conserved while the light regions are divergent, unaligned sequence. Straight end gaps indicate true sequence gaps, while squiggly line gaps indicate either unaligned sequence due to low complexity filtering or divergent sequence.

For UniGene cluster Hs.79732 representing the fibulin 1 gene, there are four subclusters represented by one consensus sequences and four exemplars. There is also a potential full length sequence which is not listed in UniGene, but is also a transcript for fibulin 1. Two probe sets represent possible alternative polyadenylation sites (top and bottom sets) while another three probe sets represent possible alternative 3′ transcript ends. (middle three probe sets).

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

What is claimed is:

1. A method of designing a nucleic acid probe array to target a transcript cluster comprising:
selecting a first set of probes comprising at least one probe targeting a first region immediately upsteam of a first polyadenylation site in the transcript cluster; and
selecting a second set of probes comprising at least one probe targeting a second region immediately upstreatm of a second polyadenylation site in the transcript cluster, wherein the first and second regions are different.

2. The method of claim 1 wherein the first and second polyadenylation sites are alternative polyadenylation sites.

3. The method of claim 2 wherein the first polyadenylation site is a putative polyadenylation site.

4. The method of claim 1 wherein the first region is within 800 bases upstream of the first polyadenylation site and the second region is within 800 bases upstream of the second polyadenylation site.

5. The method of claim 4 wherein the first region is within 600 bases upstream of the first polyadenylation site and the second region is within 600 bases upstream of the second polyadenylation site.

6. The method of claim 5 wherein the target nucleic acid represents a cluster of transcript sequences including RNA and ESTs.

7. The method of claim 6 wherein the first polyadenylation site is in a full length mRNA and the first set of probes are selected to target the full length mRNA as an exemplar sequence of the cluster.

8. The method of claim 6 wherein the first polyadenylation site is shared by a stack of sequences in the cluster and the probe are selected to target the consensus sequence of the cluster, wherein the stack of sequences comprises at least 2 sequences.

9. The method of claim 8 wherein the stack of sequences comprises at least 5 sequences.

10. The method of claim 9 wherein the stack of sequences comprises at least 8 sequences.

11. The method of claim 7, 8, 9, or 10 wherein the first and second probe sets comprises at least 5 probes.

12. The method of claim 11 wherein the first and second probe sets comprises at least 10 probes.

13. A nucleic acid probe array comprising a first set of probes comprising at least one probe targeting a first region immediately upsteam of a first polyadenylation site in a transcript cluster; and a second set of probes comprising at least one probe targeting a second region immediately upstreatm of a second polyadenylation site in the transcript cluster, wherein the first and second regions are different.

14. The array of claim 13 wherein the first and second polyadenylation sites are alternative polyadenylation sites.

15. The array of claim 14 wherein the first polyadenylation site is a putative polyadenylation site.

16. The array of claim 13 wherein the first region is within 800 bases upstream of the first polyadenylation site and the second region is within 800 bases upstream of the second polyadenylation site.

17. The array of claim 16 wherein the first region is within 600 bases upstream of the first polyadenylation site and the second region is within 600 bases upstream of the second polyadenylation site.

18. The array of claim 17 wherein the target nucleic acid represents a cluster of transcript sequences including RNA and ESTs.

19. The array of claim 18 wherein the first polyadenylation site is in a full length mRNA and the first set of probes are selected to target the full length mRNA as an exemplar sequence of the cluster.

20. The array of claim 19 wherein the first polyadenylation site is shared by a stack of sequences in the cluster and the probe are selected to target the consensus sequence of the cluster, wherein the stack of sequences comprises at least 2 sequences.

21. The array of claim 20 wherein the stack of sequences comprises at least 5 sequences.

22. The array of claim 21 wherein the stack of sequences comprises at least 8 sequences.

23. The array of claim 19, 20, 21, or 22 wherein the first and second probe sets comprises at least 5 probes.

24. The array of claim 23 wherein the first and second probe sets comprises at least 10 probes.

25. The array of claim 23 wherein the probes are immobilized at a density greater than 400 different probes/cm².

26. The array of claim 23 wherein the probes are immobilized at a density greater than 1000 different probes/cm².
